(19) United States
(12) Patent Application Publication
(10) Pub. No.: US 2024/0277906 A1
(43) Pub. Date: Aug. 22, 2024

(54) LUBRICIOUS COATINGS FOR MEDICAL DEVICES WITH ENHANCED DURABILITY

(71) Applicant: Surmodics, Inc., Eden Prairie, MN (US)

(72) Inventors: David E. Babcock, Brooklyn Park, MN (US); Syed Hossainy, Hayward, CA (US)

(21) Appl. No.: 18/405,604

(22) Filed: Jan. 5, 2024

Related U.S. Application Data
(60) Provisional application No. 63/437,294, filed on Jan. 5, 2023.

(51) Int. Cl. A61L 29/08; A61L 29/14

(52) U.S. Cl. CPC A61L 29/085; A61L 29/145; A61L 2400/10; A61L 2420/06; A61L 2420/08

(57) ABSTRACT

Embodiments herein relate to lubricious coatings for medical devices. A coated medical device is included having a substrate and a lubricious coating disposed thereon. In some embodiments, the lubricious coating can include an outer protective layer including long polymer chains. The long polymer chains can include a bonded end and a mobile free end. In some embodiments, the lubricious coating can include a hydrogel layer and long polymer chains that are at least partially disposed within the lubricious coating. Other embodiments are also included herein.

This application claims the benefit of U.S. Provisional Application No. 63/437,294, filed Jan. 5, 2023, the content of which is herein incorporated by reference in its entirety.

### FIELD

Embodiments herein relate to medical device coatings. More specifically, embodiments herein relate to lubricious coatings for medical devices.

### BACKGROUND

Medical devices include those that are chronically implanted, devices that are transitorily implanted, and those that not implanted at all. Many types of medical devices are enhanced by reducing the friction between the device and the environment that surrounds the medical device, particularly during insertion of a device. As an example, catheters are inserted, at least transitorily, into the body of a subject. Reduction of friction can lead to enhanced patient comfort, procedural ease for the care provider, reduced chances for infection, and reduced tissue disruption, amongst other benefits. One approach to reducing the friction between a medical device and the environment surrounding the medical device is to apply a lubricious coating onto the medical device.

### SUMMARY

Embodiments herein relate to lubricious coatings for medical devices. In an embodiment, a coated medical device is included having a substrate and a lubricious coating disposed thereon. The lubricious coating can include an outer protective layer including long polymer chains. The long polymer chains can include a bonded end and a mobile free end.

In an embodiment, a coated medical device is included having a substrate and a lubricious coating disposed thereon. The lubricious coating can include a hydrogel layer and long polymer chains. The long polymer chains can be at least partially disposed within the lubricious coating. In some embodiments, at least some of the long polymer chains can be configured to elute therefrom within an in vivo environment.

In an embodiment, a coated medical device is included having a substrate and a lubricious coating disposed thereon. The lubricious coating can include a base coat and a hydrogel layer disposed thereon. The hydrogel layer can be disposed in a discontinuous coverage pattern.

In an embodiment, a coated medical device is included having a substrate and a lubricious coating disposed thereon. The lubricious coating can include a base coat and a hydrogel layer disposed over the base coat. The hydrogel layer can define an exterior surface. The exterior surface can define a plurality of peaks and valleys.

In an embodiment, a coated medical device is included having a substrate and a lubricious coating disposed over the substrate. The lubricious coating can include a hydrogel layer. Discrete hydrophilic particles can be disposed on or in an exterior surface of the hydrogel layer.

In an embodiment, a coated medical device is included having a substrate and a lubricious coating disposed over the substrate. The lubricious coating can include a hydrogel layer. Hydrophilic particles can be disposed on or in an exterior surface of the hydrogel layer. The lubricious coating can further include interpolymer complexes. The interpolymer complexes can include a polymer and a thromboresistant compound.

This summary is an overview of some of the teachings of the present application and is not intended to be an exclusive or exhaustive treatment of the present subject matter. Further details are found in the detailed description and appended claims. Other aspects will be apparent to persons skilled in the art upon reading and understanding the following detailed description and viewing the drawings that form a part thereof, each of which is not to be taken in a limiting sense. The scope herein is defined by the appended claims and their legal equivalents.

### BRIEF DESCRIPTION OF THE FIGURES

Aspects may be more completely understood in connection with the following figures (FIGS.), in which:

FIG. 1 is a schematic view of a coated medical device in accordance with various embodiments herein.

FIG. 2 is a cross-sectional view of a portion of a coated medical device in accordance with various embodiments herein.

FIG. 3 is a cross-sectional view of a portion of a coated medical device in accordance with various embodiments herein.

FIG. 4 is a cross-sectional view of a portion of a coating in accordance with various embodiments herein.

FIG. 5 is a cross-sectional view of a portion of a coating in accordance with various embodiments herein.

FIG. 6 is a cross-sectional view of a portion of a coating in accordance with various embodiments herein.

FIG. 7 is a schematic view of a long polymer chain that is part of a protective layer in accordance with various embodiments herein.

FIG. 8 is a schematic view of a long polymer chain that is part of a protective layer in accordance with various embodiments herein.

FIG. 9 is a schematic view of a long polymer chain that is part of a protective layer in accordance with various embodiments herein.

FIG. 10 is a cross-sectional view of a portion of a coated medical device in accordance with various embodiments herein.

FIG. 11 is a cross-sectional view of a portion of a coated medical device in accordance with various embodiments herein.

FIG. 12 is a cross-sectional view of a portion of a coated medical device in accordance with various embodiments herein.

FIG. 13 is a cross-sectional view of a portion of a coated medical device in accordance with various embodiments herein.

FIG. 14 is a cross-sectional view of a portion of a coated medical device in accordance with various embodiments herein.

While embodiments are susceptible to various modifications and alternative forms, specifics thereof have been shown by way of example and drawings, and will be described in detail. It should be understood, however, that the scope herein is not limited to the particular aspects described. On the contrary, the intention is to cover modifications, equivalents, and alternatives falling within the spirit and scope herein.

### DETAILED DESCRIPTION

As described above, one approach to reducing the friction between a medical device and the environment surrounding the medical device is to apply a lubricious coating onto the medical device. However, with some coatings, portions of the coating can rub against the vessel wall and erode as the coated device is navigated through vessels of the body. As such, some lubricious coatings may exhibit a decrease in lubricity and a corresponding increase in friction during use.

In various embodiments herein, a protective layer can be deposited over and/or incorporated into a lubricious hydrogel layer to help maintain lubricity during use. The protective layer can include highly mobile, long chain polymers (in some embodiments un-crosslinked) either grafted onto the top surface of the hydrogel layer and/or partially crosslinked into the hydrogel layer, physically or chemically, such that rest of the long chain polymer is freely mobile.

The loose chains are highly mobile, which repels and dissipates shear and normal forces from water molecules in the space between the coated surface and the tissue of a vessel wall reducing the coefficient of friction of the coating during advancement of the coated surface relative to the tissue and rendering the lubricious coating more durable.

In addition, various embodiments herein can have thromboresistant properties. The long chains can function to create a substantial hydrodynamic radius when the coating is exposed to an aqueous environment such as when exposed to an in vivo environment. While not intending to be bound by theory, the density and the height of the hydrodynamic radius contribute to the thromboresistant properties of the resulting coating as the proteins cannot associate with the surface due to the relatively large exclusion volume that is generated. While polymers with both branched and linear chains are contemplated for use herein, branched chains can offer benefits in creating a hydrodynamic radius to repel attachment of proteins.

For example, in an embodiment a coated medical device is included having a substrate and a lubricious coating disposed thereon. The lubricious coating can include an outer protective layer including long polymer chains. The long polymer chains can include a bonded end and a mobile free end.

In various embodiments herein, un-crosslinked polymer chains can be incorporated into the hydrogel and configured to slowly elute as the hydrogel is navigated through the intravascular system to maintain lubricity during use. The elution of the chains alters the no-slip condition at the vessel wall and reduces the coefficient of friction of the coating. The eluting un-crosslinked polymer chains can be used in combination with or without protective layers as described previously.

For example, in an embodiment, a coated medical device is included having a substrate and a lubricious coating disposed thereon. The lubricious coating can include a hydrogel layer and long polymer chains. The long polymer chains can be at least partially disposed within the lubricious coating. In some embodiments, at least some of the long polymer chains can be configured to elute from the coating within an in vivo environment.

In various embodiments herein, a hydrophilic coating can be provided as a plurality of lubricious patches or islands on the substrate and/or a basecoat to maintain lubricity during use. In such embodiments, the patches of lubricious coating can effectively reduce contact points with the vessel (small patches contacting the wall rather than two continuous surfaces rubbing against each other).

For example, in an embodiment, a coated medical device is included having a substrate and a lubricious coating disposed thereon. The lubricious coating can include a base coat and a hydrogel layer disposed thereon. The hydrogel layer can be disposed in a discontinuous coverage pattern.

In an embodiment, a coated medical device is included having a substrate and a lubricious coating disposed thereon. The lubricious coating can include a base coat and a hydrogel layer disposed over the base coat. The hydrogel layer can define an exterior surface. The exterior surface can define a plurality of peaks and valleys that can effectively reduce contact points with a vessel well or other tissue.

In an embodiment, a coated medical device is included having a substrate and a lubricious coating disposed over the substrate. The lubricious coating can include a hydrogel layer. Discrete hydrophilic particles can be disposed on or in an exterior surface of the hydrogel layer.

In an embodiment, a coated medical device is included having a substrate and a lubricious coating disposed over the substrate. The lubricious coating can include a hydrogel layer. Hydrophilic particles can be disposed on or in an exterior surface of the hydrogel layer. The lubricious coating can further include interpolymer complexes. The interpolymer complexes can include a polymer and a thromboresistant compound.

Many different medical devices can be coated with coatings herein including, but not limited to, chronically implantable medical devices, transitorily implantable medical devices, and the like. Further examples of implantable medical devices are provided below. However, in various embodiments, catheters of various types and/or devices having shafts can be coated.

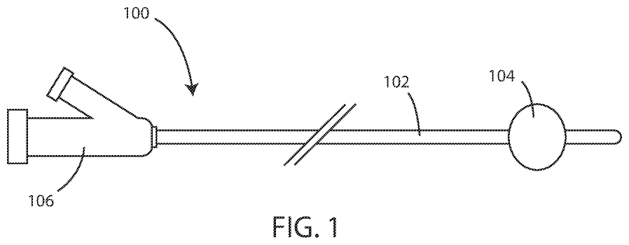

Referring now to FIG. 1, a schematic view of a coated medical device 100 is shown in accordance with various embodiments herein. It will be appreciated that the coated medical device 100 of FIG. 1 is merely one example of a medical device that can be coated with coatings described herein. In this example, the coated medical device 100 can include a catheter shaft 102, a balloon 104, and a connection manifold 106 (or proximal connector). The balloon 104 can be inflated and, in some embodiments, can also carry a drug-eluting coating. In various embodiments, the catheter shaft 102 can be coated with a coating herein. In some embodiments, the balloon 104 can be coated with a coating herein. In some embodiments, the catheter shaft 102 and the balloon 104 can be coated with a coating herein.

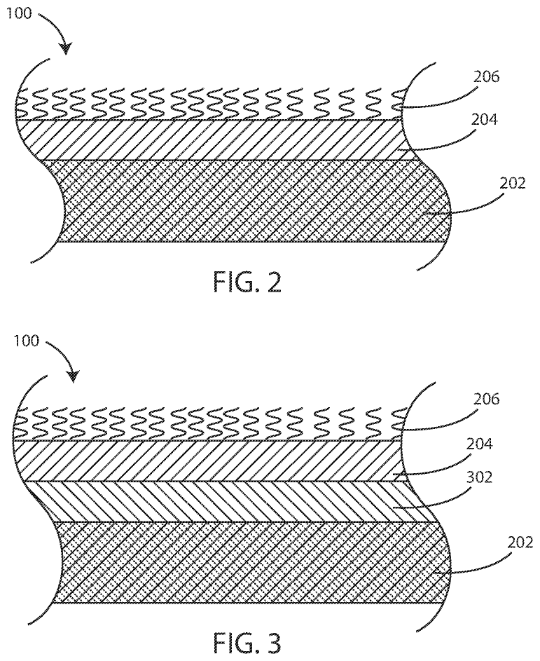

Referring now to FIG. 2, a cross-sectional view of a portion of a coated medical device 100 is shown in accordance with various embodiments herein. The coated medical device 100 includes a substrate 202. Example of substrates can include polymers, metals, ceramics, composites, and the like. In various embodiments, the substrate 202 can be at least one selected from the group consisting of a polymer and a metal. Further examples of substrates are provided below. In this example, the coated medical device 100 includes a first layer 204. The first layer 204 can be disposed over the substrate 202. In various embodiments, the first layer 204 can be a hydrogel layer. Exemplary hydrogel materials are described in greater detail below. In some embodiments, the first layer 204 can be disposed directly on the substrate 202. However, in some embodiments, an intermediate layer (such as a base coat layer) such as a tie layer can be disposed between the first layer 204 and the substrate 202.

In various embodiments, a second layer 206 (or outer layer or outer protective layer) can be disposed over the first layer 204 or hydrogel layer. The second layer 206 can include highly mobile, long chain polymers (in some embodiments un-crosslinked) either grafted onto the top surface of the first layer 204 and/or partially crosslinked into the first layer 204, physically or chemically, such that rest of the un-crosslinked portion is freely mobile. Exemplary highly mobile, long chain polymers are described in greater detail below.

In some embodiments, the second layer 206 exhibits a coefficient of friction less than the first layer 204 or hydrogel layer.

It will be appreciated that various numbers of layers can be used to form coatings herein. Referring now to FIG. 3, a cross-sectional view of a coated medical device 100 is shown in accordance with various embodiments herein. FIG. 2 is generally similar to FIG. 1. However, in this embodiment, the coated medical device 100 also includes an additional layer 302. In this case, the additional layer 302 can be disposed under the first layer 204. In some embodiments, the additional layer 302 can be in direct contact with the first layer 204. However, in some embodiments, an intermediate layer can be disposed between the first layer 204 and the additional layer 302. The first layer 204 and the additional layer 302 can be composed of the same materials and/or can be composed of different materials. In some embodiments, the additional layer 302 can be a hydrogel containing layer. In some embodiments, the additional layer 302 can be a tie layer.

The layers can be retained together in various ways. For example, in some embodiments, layers can be retained on other layers through hydrogen bonding, ionic bonding, covalent bonding, molecular entanglement, or the like.

In some embodiments, coating layers herein can be formed by applying a coating composition onto a substrate or an underlying layer. For example, a first layer can be formed by applying a first coating composition onto a substrate and, where a second layer is included, a second layer can be formed by applying a second coating composition. In some cases, the layers can be dried or otherwise cured after application of a coating composition and/or prior to applying an overlying layer. In some cases, such as where one or more components includes a photoreactive compound, actinic radiation such as UV light can be applied to the layer or layers to trigger crosslinking and/or polymerization.

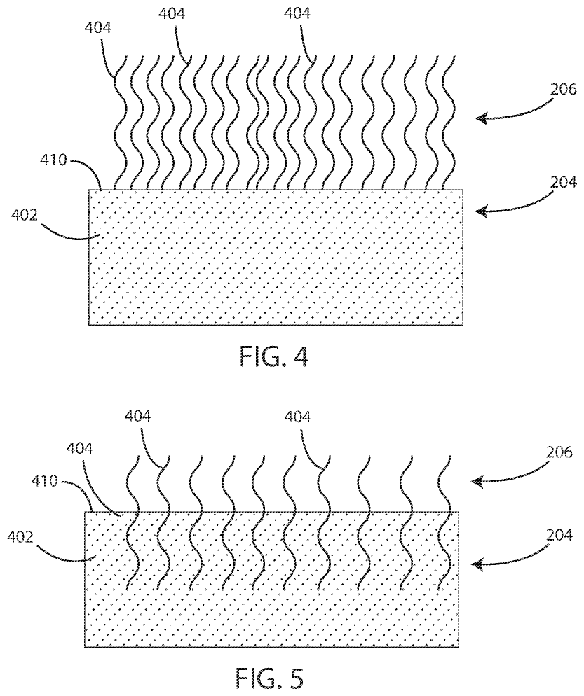

Referring now to FIG. 4, a cross-sectional view of a portion of coating is shown in accordance with various embodiments herein. FIG. 4 shows a first layer 204. The first layer 204 can include a hydrogel 402. The first layer 204 can have an outer surface 410. A second layer 206 or protective layer can be disposed over the first layer 204. The second layer 206 can include a plurality of highly mobile, long chain polymers 404 grafted onto the outer surface 410 of the first layer 204.

In some cases, a portion of the highly mobile, long chain polymers 404 can be bound into the first layer 204, such as being covalently bonded thereto, while retaining a portion that extends from the first layer 204 that is still highly mobile. Referring now to FIG. 5, a cross-sectional view of a portion of coating is shown in accordance with various embodiments herein. FIG. 5 is generally similar to FIG. 4. However, in FIG. 5 a portion of the highly mobile, long chain polymers 404 extend out of the first layer 204, while another portion is anchored within the first layer 204. In some embodiments, at least about 30, 40, 50, 60, 70, 80, 90, 95, 98, or 99 of the total length of the long chain polymers 404, or an amount falling within a range between any of the foregoing, can extend from the first layer 204.

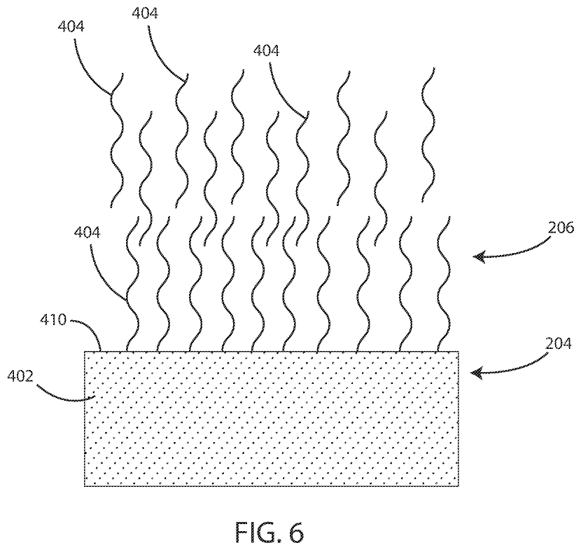

In some embodiments, a portion of the highly mobile, long chain polymers 404 may be configured to release from the surface of the first layer 204. Referring now to FIG. 6, a cross-sectional view of a portion of coating is shown in accordance with various embodiments herein. FIG. 6 is generally similar to FIG. 5 and includes a first layer 204 and a second layer 206. However, in FIG. 6 at least a portion of the highly mobile, long chain polymers 404 can release from the first layer 204. In some embodiments, at least about 10, 20, 30, 40, 50, 60, 70, 80, 90, 95, 98, 99, or 100 percent of the long chain polymers 404 are configured to release from the first layer 204, or an amount falling within a range between any of the foregoing, in vivo.

### Highly Mobile, Long Chain Polymers

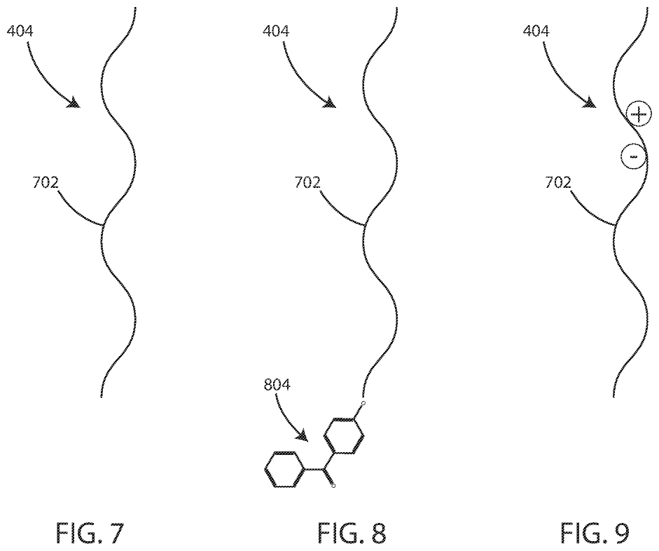

FIG. 7 is a schematic view of a long chain polymer compound 404 that is part of a protective layer in accordance with various embodiments herein. As used herein, the term long polymer chain or long chain shall refer to an average chain length greater than 50 kDa. References herein to an average with respect to chain length shall refer to number average (versus weight average) unless explicitly stated otherwise or the context dictates otherwise. The long chain polymer compound 404 can include a polymer chain portion 702. In some embodiments, the long chain polymer compound 404 can have an average chain length greater than 100 kDa, 150 kDa, 200 kDa, or more. In some embodiments, the long polymer chains have an average chain length from 100 to 300 kDa. In some embodiments, the long polymer chains are linear. However, in many embodiments the long polymer chains are branched. In various embodiments, the long polymer chains can include a comb structure, a bottle brush structure, a star structure, a hyperbranched structure, a mucin structure, and/or a dendritic structure. While the long polymer chains can be cross-linked, in many embodiments the long polymer chains are uncross-linked.

The long polymer chains can include homopolymers, copolymers, and terpolymers. In some embodiments, the long polymer chains can include at least one selected from the group consisting of a polyvinylpyrrolidone, a polyvinylalcohol, a polyacrylamide, a polyethyleneglycol, a polysulfobetaine, a phosphoryl choline capped polymer, and a poloxamer.

The long polymer chains can include a bonded end and a mobile free end. In some embodiments, the long polymer chains can be configured as a random coil.

In various embodiments, the long polymer chains are grafted onto an underlying surface or at least partially embedded within and crosslinked into an underlying layer.

In some embodiments, the long chain polymer compound 404 can include one or more functional groups to facilitate binding or grafting of the compound 404 to a surface, such as a surface of the first layer herein. Referring now to FIG. 8, a schematic view is shown of a long polymer chain that is part of a protective layer in accordance with various embodiments herein. In this example, the long chain polymer compound 404 can include a photoactivatable group 804, such as a benzophenone group. Other groups can also be used to facilitate grafting or other attachment of the long chain polymer compound 404 to a surface or within a hydrogel or other polymer matrix.

In various embodiments, the long chain polymer compound 404 can include a polymer chain portion 702 that is uncharged. However, in some embodiments, the polymer chain portion 702 can include functional groups thereon carrying a charge such that the polymer chain portion 702 is anionic or cationic. In some embodiments, the polymer chain portion 702 can be zwitterionic. The long chain polymers can specifically include a zwitterionic subunit. The zwitterionic subunit can be disposed on an exterior portion of the long chain polymers. In some embodiments the zwitterionic subunit can be at or adjacent to an end of the long chain polymers. Referring now to FIG. 9, a schematic view of a long chain polymer that is part of a protective layer is shown in accordance with various embodiments herein. In this embodiment, the polymer chain portion 702 is zwitterionic.

In various embodiments the long chain polymers can include a non-ionic backbone chain. In some embodiments, the non-ionic backbone chain comprising a degradable linkage. An exemplary degradable linkage can include an alpha-hydroxy ester linkage.

In some embodiments, at least some of the long chain polymers can be non-covalently bonded. In some cases, the non-covalently bonded long chain polymers can be configured to elute from the lubricious coating.

### Further Embodiments

In various embodiments herein, a hydrophilic coating can be provided on the substrate and/or a basecoat as a plurality of lubricious patches or islands to maintain lubricity during use. In such embodiments, the islands of lubricious coating can effectively reduce contact points with the vessel wall (small patches contacting the wall rather than two continuous surfaces rubbing against each other).

In some embodiments, the lubricious patches can be formed of a hydrogel and, as such, a hydrogel layer herein can be disposed in a discontinuous coverage pattern. In some embodiments, the hydrogel layer covers from 20 to 95 percent of the surface area of a total area covered by the lubricious coating. In some embodiments, the hydrogel layer covers from 40 to 80 percent of the surface area of a total area covered by the lubricious coating.

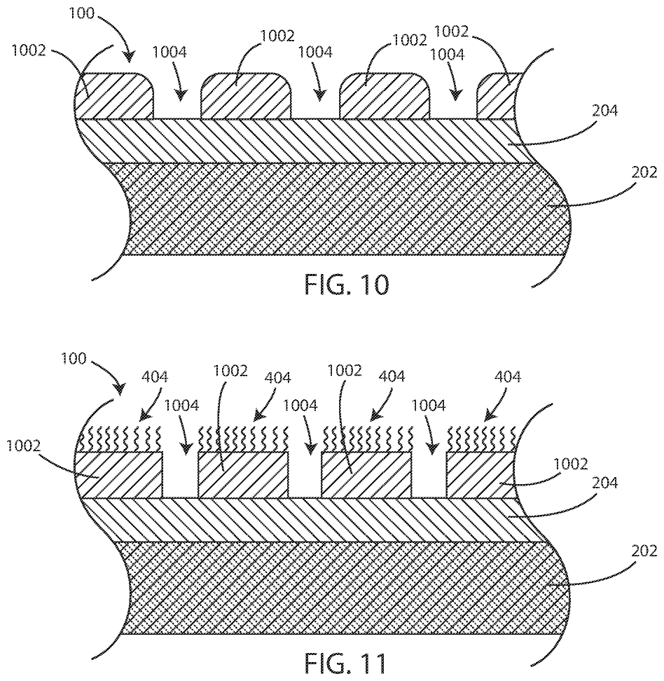

Referring now to FIG. 10, a cross-sectional view of a portion of a coated medical device 100 is shown in accordance with various embodiments herein. The coated medical device 100 can include a substrate 202 and a first layer 204 disposed on the substrate. The coated medical device 100 can also include a plurality of lubricious patches 1002 disposed on the first layer 204 with gaps 1004 between adjacent lubricious patches 1002. The lubricious patches 1002 can be of various sizes. The patches 1002 can have various shapes as viewed from the top. In some embodiments, the patches 1002 can be roughly circular, square, rectangular, polygonal, or irregular. In some embodiments, the lubricious patches 1002 can be formed of a hydrogel material, such as those described herein. In some embodiments the lubricious patches 1002 can be formed using a masking technique, such as masking the areas representing the gaps 1004 and then applying a coating composition to create the lubricious patches 1002. In some embodiments, a photolithography technique can be used to form the lubricious patches 1002.

In some embodiments, highly mobile, long chain polymers can be disposed on the lubricious patches 1002. Referring now to FIG. 11, a cross-sectional view of a portion of a coated medical device 100 is shown in accordance with various embodiments herein. The coated medical device 100 can include a substrate 202 and a first layer 204 disposed on the substrate. The coated medical device 100 can also include a plurality of lubricious patches 1002 disposed on the first layer 204 with gaps 1004 between adjacent lubricious patches 1002. Long chain polymers 404 can be disposed on top of the lubricious patches 1002.

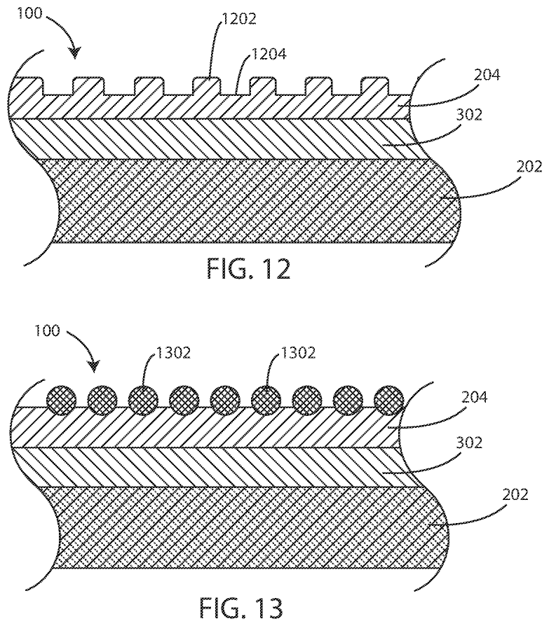

In some embodiments, a lubricious coating layer can have peaks and valleys to reduce contact points with the vessel wall, but without the gaps as described with respect to FIG. 10. Referring now to FIG. 12, a cross-sectional view of a portion of a coated medical device 100 is shown in accordance with various embodiments herein. The coated medical device 100 can include a substrate 202 and, optionally, a base layer or additional layer 302 disposed on the substrate 202 along with a first layer 204 disposed on the additional layer 302 or directly on the substrate 202 when a base layer or additional layer 302 is not included. The coated medical device 100 can also include a lubricious material layer 1002 disposed on the first layer 204 with peaks 1202 and valleys 1204 to effectively reduce the surface area of contact with a vessel wall. In various embodiments, the lubricious material layer 302 can include hydrogel materials as described herein.

In some embodiments, a coated device herein can include a coating with discrete particles disposed on an outer surface thereof. The particles can be hydrophilic particles and can be disposed on or in an exterior surface of the hydrogel layer. Referring now to FIG. 13, a cross-sectional view of a portion of a coated medical device 100 is shown in accordance with various embodiments herein. As before, the coated medical device 100 can include a substrate 202 and, optionally, a base layer or additional layer 302 disposed on the substrate 202 along with a first layer 204 disposed on the additional layer 302 or directly on the substrate 202 when a base layer or additional layer 302 is not included. Particles 1032 can be disposed on or in the surface of the first layer 204.

In various embodiments the particles 1032 can be PVP particles. In various embodiments the particles 1032 can be crosslinked PVP particles. In various embodiments the particles 1032 can be cross-linked into the hydrogel layer. In various embodiments the particles 1032 can be secured to the hydrogel layer via hydrogen bonding. In various embodiments the particles 1032 can be at least partially entangled with polymers of the hydrogel layer. In various embodiments the particles 1032 can have an average diameter that is greater than the thickness of the first layer 204, which can be a hydrogel layer including one or more of polyvinylpyrrolidone (PVP), a photo-reactive PVP, polyacrylamide (PA), and a photo-reactive PA.

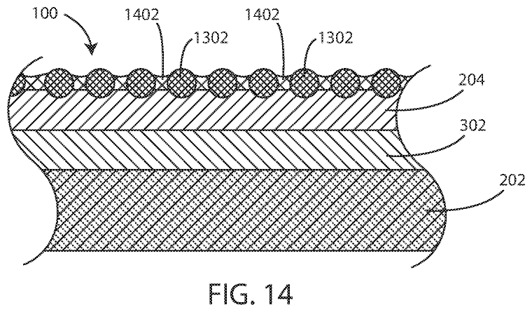

In some embodiments, an interpolymer complex can be disposed between particles. Referring now to FIG. 14, a cross-sectional view of a portion of a coated medical device 100 is shown in accordance with various embodiments herein. FIG. 14 is generally similar to FIG. 13. However, FIG. 14 also includes interpolymer complexes 1402 between particles 1302. The interpolymer complexes 1402 can include a polymer and a thromboresistant compound. A thromboresistant compound is a compound that inhibits one or more of protein and cell adsorption, thrombin and fibrin formation, and platelet activation and aggregation. Exemplary thromboresistant compounds can include heparin compounds or a derivative thereof, albumin, phosphorylcholine, and the like. The polymer of the interpolymer complexes 1402 can include at least one selected from the group consisting of polyacrylic acid (PAA), polyethylene oxide (PEO), and/or PAA/PEO copolymers, and non-ionic polymers. In some embodiments, the polymer of the interpolymer complexes 1402 can be crosslinked.

### Methods

Many different methods are contemplated herein, including, but not limited to, methods of making, methods of using, and the like. Specifically, in some embodiments a method of making a coated medical device is included herein. The method can include obtaining a substrate. The method can further include applying a first layer of a coating over the substrate. The first layer of the coating can include a hydrogel. The method can further include applying a second layer over the first layer. In some embodiments, the second layer can be a protective layer. The second layer can include highly mobile, long chain polymers.

Coating can be performed using various techniques including, but not limited to, spray coating, dip coating, blade coating, printing, contact coating techniques, and the like. Some methods herein can include one or more operations of drying or otherwise curing. Some methods herein can include one or more operations of applying actinic radiation, such as UV light, to the applied layer or layers.

### Hydrogels

In various embodiments herein, one or more layers of a coating can include a hydrogel. Hydrogels are hydrophilic polymers, with a three-dimensional network structure that has the ability to absorb a large volume of water due to the presence of hydrophilic moieties. Hydrogels herein can include both natural and synthetic hydrogels. Hydrogels herein can those that are homopolymers, copolymers, semi-interpenetrating networks, and interpenetrating networks. Hydrogels herein can include those with a cross-linked or uncrosslinked network structure. Hydrogels herein can include those that are charged (cationic and/or anionic groups) and those that are uncharged. Hydrogels herein can include those that are amorphous, semicrystalline, crystalline, and those that are hydrocolloid aggregates.

Hydrogels herein can include, but are not limited to, homopolymer or copolymers of polyethylene glycol (PEG), PEG-PEGMA, polyacrylic acid (PAA), methacrylic acid (MAA), polyethylene oxide (PEO), polyvinyl alcohol (PVA), polyacrylamide, 2-hydroxyethyl methacrylate (HEMA), carboxymethyl cellulose (CMC), polyvinylpyrrolidone (PVP), poly(N-isopropylacrylamide (PNIPAM), chitosan, acrylate-modified PEG, acrylate-modified hyaluronic acid, and the like. In some embodiments, a hydrogel herein can include one or more of polyvinylpyrrolidone (PVP), a photo-reactive PVP (e.g., a PVP compound including a photoreactive group such as a benzophenone), polyacrylamide (PA), and a photo-reactive PA (e.g., a PA compound including a photoreactive group such as a benzophenone).

In some embodiments, hydrogels herein can be crosslinked. For example, a crosslinking agent can be used to crosslink the hydrogel. Exemplary crosslinking agents are described in greater details elsewhere herein. However, in some embodiments the hydrogel can be un-crosslinked. In some embodiments, a hydrogel or hydrogel layer herein can include a first sublayer and a second sublayer. The first and second sublayers can be the same or different. In some embodiments, the second sublayer can be crosslinked to a greater extent than the first sublayer.

Some exemplary hydrogel layers are described in U.S. Pat. Nos. 9,375,517; 9,737,639; and 10,905,802; and U.S. Pat. Publ. No. 2021/0220525, the content of all of which is herein incorporated by reference.

### Polyzwitterion Compounds (Zwitterionic Polymers)

Some embodiments herein can include one or more polyzwitterion compounds. Polyzwitterion compounds herein can include polymers including both anionic and cationic groups thereon and, more specifically, any polymer in which the monomers are zwitterions.

Polyzwitterion compounds herein can include, but are not limited to, homopolymers and copolymers including polyphosphobetaines, polysulfobetaines, polycarbobetaines, polyphosphocholines, polytrimethylamine N-oxide, polyectoine, poly(3-(N-(2-(methacryloyloxy)ethyl)-N,N-dimethylammonio)propanesulfonate), poly-N-(carboxymethyl)-N,N-dimethyl-2-(methacryloyloxy) ethanaminium) (PCDME)

### Polyvinylpyrrolidone Compounds

Various embodiments herein include a polyvinylpyrrolidone homopolymer and/or copolymer (photo derivatized or not). Further details about exemplary polyvinylpyrrolidone polymers (homopolymers and copolymers) are provided as follows. However, it will be appreciated that this is merely provided by way of example and that further variations are contemplated herein.

Polyvinylpyrrolidone polymers herein can include polyvinylpyrrolidone homopolymers as well as polyvinylpyrrolidone subunit containing copolymers. By way of example, polyvinylpyrrolidone copolymers can include subunits of polyvinylpyrrolidone as follows:

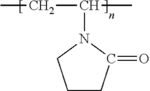

In various embodiments, a PVP copolymer can include at least one including at least one of PVP-co-PEO (polyvinylpyrrolidone-co-polyethylene oxide) and PVP-co-PSB (polyvinylpyrrolidone-co-polysulfobetaine), PVP-co-PAA, and photo-PVP-co-PAA (a photo-derivatized PVP such as PVP modified to include a benzophenone functional group or another photoactivatable functional group).

Polyvinylpyrrolidone polymers herein can be linear or can be branched. Polyvinylpyrrolidone polymers herein can have various molecular weights such as an average molecular weight from 1 kDa to 3000 kDa.

In various embodiments, the polyvinylpyrrolidone component is a blend of different molecular weight PVP compounds. Exemplary non-photo derivatized polyvinylpyrrolidone polymers can include, for example, PVP K12, PVP K30, PVP K90, and the like.

Polyvinylpyrrolidone polymers herein can be used to form a PVP hydrogel.

In various embodiments, the polyvinylpyrrolidone can include a non-photoreactive polyvinylpyrrolidone. However, instead of or in addition to non-photoreactive polyvinylpyrrolidone, in various embodiments, the polyvinylpyrrolidone can also include a photoreactive polyvinylpyrrolidone (“photo-PVP”). Photoreactive polyvinylpyrrolidones can include homopolymers and/or copolymers where they are derivatized to include a photoreactive group. In various embodiments, the photoreactive polyvinylpyrrolidone can specifically include a benzophenone group.

An exemplary photoreactive polyvinylpyrrolidone copolymer can include poly[vinyl pyrrolidone-co-N-(3-(4-benzoylbenzamideo)propyl)methacrylamide] (or PVP-co-APMA with 80 to 99.9 mole percent PVP and 20 to 0.1 mole percent APMA). By way of example, an exemplary photoreactive polyvinylpyrrolidone is as follows:

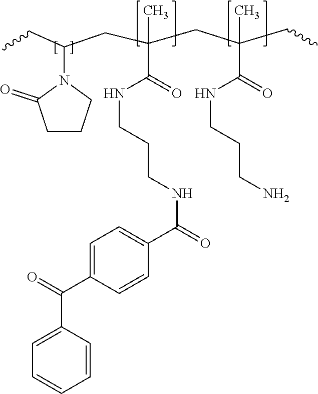

Another exemplary photoreactive polyvinylpyrrolidone copolymer (acetylated PVP-APMA-BBA; or acetylated photo-PVP) is as follows:

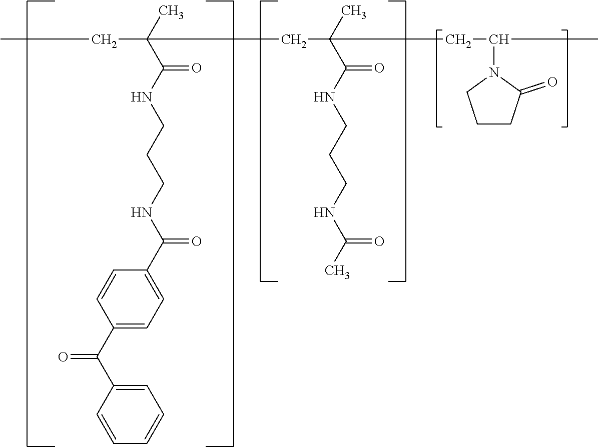

This compound can be prepared by a copolymerization of 1-vinyl-2-pyrrolidone and N-(3-aminopropyl)methacrylamide (APMA), followed by photoderivatization of the polymer using 4-benzoylbenzoyl chloride under Schotten-Baumann conditions. The unreacted amines of the photopolymer can be further acetylated using acetic anhydride. The polyvinylpyrrolidone used herein can be of various molecular weights. In some embodiments, a non-photoreactive polyvinylpyrrolidone herein can have an average molecular weight from 1 kDa to 3000 kDa. In various embodiments, a non-photoreactive polyvinylpyrrolidone can have an average molecular weight from 10 kDa to 50 kDa. In various embodiments, a non-photoreactive polyvinylpyrrolidone can be a blend of different molecular weight PVP compounds. For example, in various embodiments, the second non-photoreactive polyvinylpyrrolidone of the second layer can be a blend of at least two different molecular weight average PVP compositions.

In various embodiments, a weight ratio of a branched PVP to a poly(acrylic acid) in the same layer or a different layer is from 70:100 to 10:90.

### Crosslinking Agents

Various embodiments herein include a crosslinking agent in one or more layers of the coating. For example, in some embodiments, hydrogel layers herein can also include one or more crosslinking agents. Further details about exemplary crosslinking agents are provided as follows. However, it will be appreciated that this is merely provided by way of example and that further variations are contemplated herein.

In some embodiments, the crosslinking agent(s) can have a molecular weight of less than about 1500 kDa, but in other embodiments can be larger. In some embodiments the crosslinking agent can have a molecular weight of less than about 1200, 1100, 1000, 900, 800, 700, 600, 500, or 400 or less, or a molecular weight falling within a range between any of the foregoing.

In various embodiments, cross-linking agents include one or more photoreactive groups attached to a linking group. The cross-linking agent (or linking agent) can be represented by the formula Photo¹-LG-Photo², wherein Photo¹ and Photo² independently represent at least one photoreactive group and LG represents a linking group. The term “linking group” as used herein, refers to a segment or group of molecules configured to connect two or more molecule to each another. In some embodiments, the linking group can include a heteroatom. In some embodiments, the linking group lacks a heteroatom. In one embodiment, the linking group includes at least one silicon atom. In another embodiment, the linking group includes at least one phosphorus atom.

In some embodiments, the linking group can be a degradable linking group, which in other embodiments the linking group can be a non-degradable linking group. The term “degradable linking group” as used herein, refers to a moiety configured to connect one molecule to another, wherein the linking group is capable of cleavage under one or more conditions. The term “biodegradable” as used herein, refers to degradation in a biological system, and includes for example, enzymatic degradation or hydrolysis. It should be noted that the term “degradable” as used herein includes both enzymatic and non-enzymatic (or chemical) degradation. It is also understood that hydrolysis can occur in the presence of or without an acid or base. In one embodiment, the linking agent is water soluble. In another embodiment, the linking agent is not water soluble.

In various embodiments the linking group can function as a spacer, for example, to increase the distance between the photoreactive groups of the linking agent. For example, in some instances it may be desirable to provide a spacer to reduce steric hindrance that may result between the photoreactive groups, which could interfere with the ability of the photoreactive groups to form covalent bonds with a support surface, or from serving as a photoinitiator for polymerization. As described herein, it is possible to vary the distance between the photoreactive groups, for example, by increasing or decreasing the spacing between one or more photoreactive groups.

As described herein, one or more photoreactive groups can be bound to a linking group by a degradable or a non-degradable linkage. In various embodiments, the degradable linkage between the photoreactive group and the linking group includes at least one heteroatom, including, but not limited to oxygen, nitrogen, selenium, sulfur or a combination thereof. In one embodiment, a photoreactive group, linking group and heteroatom form an ether (R¹—O—R²), wherein R¹ is a photoreactive group and R² is a linking group. In another embodiment, a photoreactive group, linking group and heteroatom form an amine,

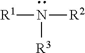

wherein R¹ is a photoreactive group, R² is a linking group, and R³ is hydrogen, aryl or alkyl, a photoreactive group, or a hydroxyl or salt thereof. In one embodiment, R³ is cyclic, linear or branched, saturated or unsaturated, aromatic or heteroaromatic, or a combination thereof. The stability of the ether and/or amine linkage can be influenced depending upon the size (e.g., chain length, branching, bulk, etc.) of the substituents. For example, bulkier substituents will generally result in a more stable linkage (i.e., a linking agent that is slower to degrade in the presence of water and/or acid).

In various embodiments, the linking group includes one or more silicon atoms. In a particular embodiment, the linking group includes one silicon atom (which can be referred to as a monosilane) covalently bound to at least two photoreactive groups. In another embodiment, the linking group includes at least two silicon atoms (which can be referred to as a disilane). In one embodiment, the linking group can be represented by the formula Si—Y—Si, wherein Y represents a linker that can be null (e.g., the linking group includes a direct Si—Si bond), an amine, ether, linear or branched C₁-C₁₀ alkyl, or a combination thereof. In one embodiment, Y is selected from O, CH₂, OCH₂CH₂O and O(CH₂CH₂O)ₙ, wherein n is an integer between 1 and 5, between 1 and 10, between 1 and 15, between 1 and 20, between 1 and 25, or between 1 and 30. One embodiment of a disilane linking agent is shown below

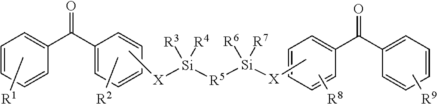

wherein R¹, R², R⁸ and R⁹ can be any substitution, including, but not limited to H, alkyl, halide, hydroxyl, amine, or a combination thereof; R³, R⁴, R⁶ and R⁷ can be alkyl, aryl or a combination thereof; R⁵ can be any substitution, including but not limited to O, alkyl or a combination thereof; and each X, independently, can be O, N, Se, S, or alkyl, or a combination thereof. One specific embodiment is shown below:

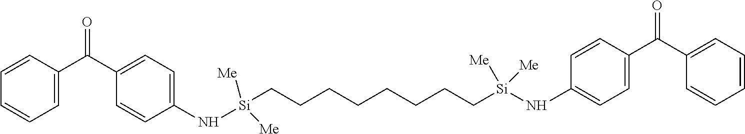 [SMILES: C[Si](C)(CCCCCCCC[Si](C)(C)Nc1ccc(C(=O)c2ccccc2)cc1)Nc1ccc(C(=O)c2ccccc2)cc1]

In various embodiments, the linking agent can be represented by the formula

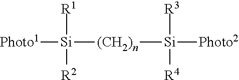

wherein Photo¹ and Photo², independently, represent one or more photoreactive groups and n is an integer between 1 and 10, wherein the linking agent comprises a covalent linkage between at least one photoreactive group and the linking group, wherein the covalent linkage between at least one photoreactive group and the linking group is interrupted by at least one heteroatom. In general, a longer hydrocarbon chain between the two silicon atoms will tend to increase the flexibility of the linking agent and may facilitate crosslinking between a greater number of polymers than a linking agent with a shorter carbon chain, since the photoreactive groups can react with polymers located farther apart from one another. In the formula shown above, R¹, R², R³, R⁴ are independently alkyl or aryl, including, but not limited to cyclic, linear or branched, saturated or unsaturated, aromatic or heteroaromatic, or a combination thereof. In a more particular embodiment, R¹-R⁴ are independently phenyl, methyl, ethyl, isopropyl, t-butyl, or a combination thereof. In another embodiment, R¹-R⁴ can also be, independently, a photoreactive group. In yet another embodiment, R¹-R⁴ can also be, independently, hydroxyl or salt thereof. In one embodiment, the hydroxyl salt includes a counterion that is lithium, sodium, potassium, or a combination thereof.

In another embodiment, the linking agent can be represented by the formula

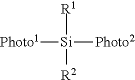

wherein Photo¹ and Photo², independently, represent one or more photoreactive group, wherein the linking agent comprises a covalent linkage between at least one photoreactive group and the linking group, wherein the covalent linkage between at least one photoreactive group and the linking group is interrupted by at least one heteroatom; R¹ and R² are independently alkyl or aryl, including, but not limited to cyclic, linear or branched, saturated or unsaturated, aromatic or heteroaromatic, or a combination thereof. In a more particular embodiment, R¹ and R² are independently phenyl, methyl, ethyl, isopropyl, t-butyl, or a combination thereof. R¹ and R² can also be, independently, a photoreactive group, wherein the linking agent comprises a covalent linkage between at least one photoreactive group and the linking group, wherein the covalent linkage between at least one photoreactive group and the linking group is interrupted by at least one heteroatom; or hydroxyl or salt thereof. In one embodiment, the hydroxyl salt includes a counterion that is lithium, sodium, potassium, or a combination thereof. One embodiment of a monosilane linking agent is shown below

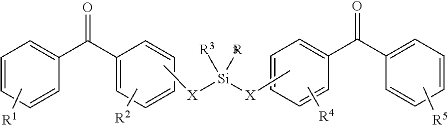

in which R¹ and R⁵ can be any substitution, including, but not limited to H, halogen, amine, hydroxyl, alkyl, or a combination thereof; R² and R⁴ can be any substitution, except OH, including, but not limited to H, alkyl or a combination thereof; R³ can be alkyl, aryl or a combination thereof, including, for example, methyl, ethyl, propyl, isopropyl and butyl; and X, independently, can be O, N, Se, S, alkyl or a combination thereof.

In another embodiment, the linking group includes one or more phosphorous atoms. In one embodiment, the linking group includes one phosphorus atom (which can also be referred to as a mono-phosphorus linking group). In another embodiment, the linking agent includes two phosphorus atoms (which can also be referred to as a bis-phosphorus linking group). In one embodiment, the linking group comprises at least one phosphorus atom with a phosphorus-oxygen double bond (P═O), wherein at least one or two photoreactive groups are bound to the phosphorus atom. In another embodiment, the linking group comprises one phosphorus atom with a phosphorus-oxygen double bond (P═O), wherein two or three photoreactive groups are covalently bound to the phosphorus atom. In another embodiment, the linking group comprises at least two phosphorus atoms, wherein at least one phosphorus atom includes a phosphorus-oxygen double bond (P═O), and at least one or two photoreactive groups are covalently bound to each phosphorus atom.

In a more particular embodiment, the linking agent can be represented by the formula:

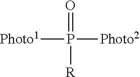

wherein Photo¹ and Photo², independently, represent one or more photoreactive groups, wherein the linking agent comprises a covalent linkage between at least one photoreactive group and the linking group, wherein the covalent linkage between at least one photoreactive group and the linking group is interrupted by at least one heteroatom and R is alkyl or aryl, a photoreactive group, hydroxyl or salt thereof, or a combination thereof. In one embodiment, the hydroxyl salt includes a counterion that is lithium, sodium, potassium, or a combination thereof. In a more particular embodiment, R is cyclic, linear or branched, saturated or unsaturated, aromatic or heteroaromatic, or a combination thereof. In a more particular embodiment, R is phenyl, methyl, ethyl, isopropyl, t-butyl, or a combination thereof.

In another embodiment, the linking agent can be represented by formula:

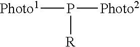

wherein Photo¹ and Photo² independently, represent one or more photoreactive groups, wherein the linking agent comprises a covalent linkage between at least one photoreactive group and the linking group, wherein the covalent linkage between at least one photoreactive group and the linking group is interrupted by at least one heteroatom and R is alkyl or aryl, a photoreactive group (wherein the covalent linkage between the photoreactive group and the linking group may be interrupted by at least one heteroatom), hydroxyl or salt thereof, or a combination thereof. In one embodiment, the hydroxyl salt includes a counterion that is lithium, sodium, potassium, or a combination thereof. In a more particular embodiment, R is cyclic, linear or branched, saturated or unsaturated, aromatic or heteroaromatic, or a combination thereof. In one embodiment, R is phenyl, methyl, ethyl, isopropyl, t-butyl, or a combination thereof.

In another embodiment, the linking agent can be represented by the formula:

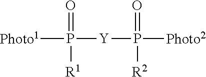

wherein Photo¹ and Photo², independently, represent one or more photoreactive groups, wherein the linking agent comprises a covalent linkage between at least one photoreactive group and the linking group, wherein the covalent linkage between at least one photoreactive group and the linking group is interrupted by at least one heteroatom; Y represents a linker that can be null (i.e., not present, such that the linking group includes a direct P—P bond), Nor O, linear or branched C₁-C₁₀ alkyl, or a combination thereof; and R¹ and R² are independently alkyl, aryl, a photoreactive group (wherein the covalent linkage between the photoreactive group and the linking group can be interrupted by at least one heteroatom), hydroxyl or salt thereof, or a combination thereof. In one embodiment, Y is selected from O, CH₂, OCH₂O, OCH₂CH₂O and O(CH₂CH₂O)ₙ, wherein n is an integer between 1 and 5, between 1 and 10, between 1 and 15, between 1 and 20, between 1 and 25, or between 1 and 30. In one embodiment, the hydroxyl salt counterion is lithium, sodium, potassium, or a combination thereof. In a more particular embodiment, R¹ and R² are independently, cyclic, linear or branched hydrocarbon, saturated or unsaturated, aromatic or heteroaromatic, or a combination thereof. In one embodiment, R¹ and R² are independently phenyl, methyl, ethyl, isopropyl, t-butyl, or a combination thereof. In general, a longer hydrocarbon chain between the two phosphorus atoms will tend to increase the flexibility of the linking agent and may facilitate crosslinking between a greater number of polymers than a linking agent with a shorter carbon chain, since the reactive photoreactive groups can react with polymers located farther apart from one another. In one embodiment, Y can be O, CH₂, OCH₂CH₂O and O(CH₂CH₂O)ₙ wherein n is an integer between 1 and 5, between 1 and 10, between 1 and 15, between 1 and 20, between 1 and 25, or between 1 and 30. One embodiment is shown below

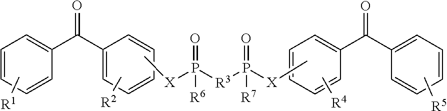

in which R¹, R², R⁴ and R⁵ can be any substitution, including but not limited to H, alkyl, halogen, amine, hydroxyl, or a combination thereof; R³ can be any substitution, including but not limited to O, alkyl, or a combination thereof; and each X can independently be O, N. Se, S, alkyl, or a combination thereof. In one embodiment, the linking agent includes one or more phosphoester bonds and one or more phosphoramide bonds, and can be represented by the formula:

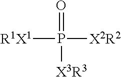

wherein X and X² are, independently, O, N, Se, S or alkyl; R¹ and R² are independently, one or more photoreactive groups, and X³ is O, N, Se, S, alkyl or aryl; R³ is alkyl or aryl, including, but not limited to cyclic, linear or branched, saturated or unsaturated, aromatic or heteroaromatic, or a combination thereof. In a more particular embodiment, R³ is phenyl, methyl, ethyl, isopropyl, t-butyl, or a combination thereof. R³ can also be a photoreactive group or a hydroxyl or salt thereof. In one embodiment, the hydroxyl salt counterion is lithium, sodium, potassium, or a combination thereof.

In one embodiment, the linking agent comprises a triphosphorester, which can be represented by the formula.

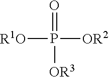

wherein R¹ and R² are independently, one or more photoreactive groups, and R³ is alkyl or aryl, including, but not limited to cyclic, linear or branched, saturated or unsaturated, aromatic or heteroaromatic, or a combination thereof. In a more particular embodiment, R³ is phenyl, methyl, ethyl, isopropyl, t-butyl, or a combination thereof. R³ can also be a photoreactive group or hydrogen, or a hydroxyl salt. In one embodiment, the hydroxyl salt counterion is lithium, sodium, potassium, or a combination thereof.

Some specific embodiments include the following linking agents:
(a) bis(4-benzoylphenyl) hydrogen phosphate:

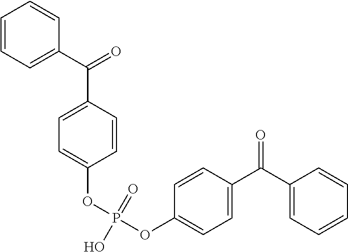 [SMILES: O=C(c1ccccc1)c1ccc(OP(=O)(O)Oc2ccc(C(=O)c3ccccc3)cc2)cc1]

(b) sodium bis(4-benzoylphenyl phosphate):

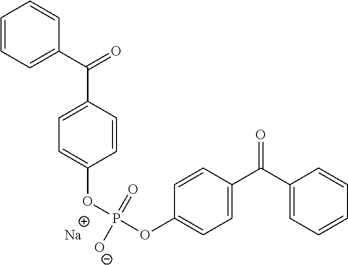 [SMILES: O=C(c1ccccc1)c1ccc(OP(=O)([O-])Oc2ccc(C(=O)c3ccccc3)cc2)cc1 ; [Na+]]

(c) tris(4-benzyolphenyl) phosphate):

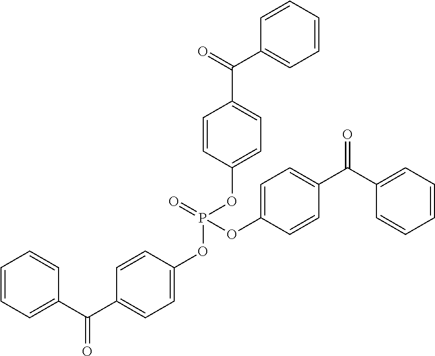 [SMILES: O=C(c1ccccc1)c1ccc(OP(=O)(Oc2ccc(C(=O)c3ccccc3)cc2)Oc2ccc(C(=O)c3ccccc3)cc2)cc1]

(d) tetrakis(4-benzoylphenyl)methylenebis(phosphonate)

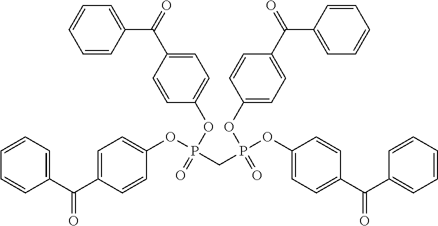 [SMILES: O=C(c1ccccc1)c1ccc(OP(=O)(CP(=O)(Oc2ccc(C(=O)c3ccccc3)cc2)Oc2ccc(C(=O)c3ccccc3)cc2)Oc2ccc(C(=O)c3ccccc3)cc2)cc1]

In another embodiment, the linking agent comprises a triphosphoramide, which can be represented by the formula.

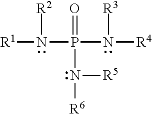

wherein R¹-R⁶ are independently, a photoreactive group, a hydroxyl or salt thereof, alkyl or aryl, or a combination thereof, wherein at least two of R¹-R⁶ are, independently, a photoreactive group. In one embodiment, the hydroxyl salt counterion is lithium, sodium, potassium, or a combination thereof. In a more particular embodiment, R¹-R⁶ are independently cyclic, linear or branched, saturated or unsaturated, aromatic or heteroaromatic, or a combination thereof. In a more particular embodiment, R¹-R⁶ are, independently, phenyl, methyl, ethyl, isopropyl, t-butyl, or a combination thereof.

In some embodiments, the photoactivatable cross-linking agent can be ionic, and can have good solubility in an aqueous composition, such as the first and/or second coating composition. Thus, in some embodiments, at least one ionic photoactivatable cross-linking agent is used to form the coating. In some cases, an ionic photoactivatable cross-linking agent can crosslink the polymers within the second coating layer which can also improve the durability of the coating.

Any suitable ionic photoactivatable cross-linking agent can be used. In some embodiments, the ionic photoactivatable cross-linking agent is a compound of formula I: X₁—Y—X₂ where Y is a radical containing at least one acidic group, basic group, or a salt of an acidic group or basic group. X₁ and X₂ are each independently a radical containing a latent photoreactive group. The photoreactive groups can be the same as those described herein. Spacers can also be part of X₁ or X₂ along with the latent photoreactive group. In some embodiments, the latent photoreactive group includes an aryl ketone or a quinone.

The radical Y in formula I provides the desired water solubility for the ionic photoactivatable cross-linking agent. The water solubility (at room temperature and optimal pH) is at least about 0.05 mg/ml. In some embodiments, the solubility is about 0.1 to about 10 mg/ml or about 1 to about 5 mg/ml.

In some embodiments of formula I, Y is a radical containing at least one acidic group or salt thereof. Such a photoactivatable cross-linking agent can be anionic depending upon the pH of the coating composition. Suitable acidic groups include, for example, sulfonic acids, carboxylic acids, phosphonic acids, and the like. Suitable salts of such groups include, for example, sulfonate, carboxylate, and phosphate salts. In some embodiments, the ionic cross-linking agent includes a sulfonic acid or sulfonate group. Suitable counter ions include alkali, alkaline earths metals, ammonium, protonated amines, and the like.

For example, a compound of formula I can have a radical Y that contains a sulfonic acid or sulfonate group; X₁ and X₂ can contain photoreactive groups such as aryl ketones. Such compounds include 4,5-bis(4-benzoylphenylmethyleneoxy)benzene-1,3-disulfonic acid or salt; 2,5-bis(4-benzoylphenylmethyleneoxy)benzene-1,4-disulfonic acid or salt; 2,5-bis(4-benzoylmethyleneoxy)benzene-1-sulfonic acid or salt; N,N-bis[2-(4-benzoylbenzyloxy)ethyl]-2-aminoethanesulfonic acid or salt, and the like. See U.S. Pat. No. 6,278,018. The counter ion of the salt can be, for example, ammonium or an alkali metal such as sodium, potassium, or lithium.

In other embodiments of formula I, Y can be a radical that contains a basic group or a salt thereof. Such Y radicals can include, for example, an ammonium, a phosphonium, or a sulfonium group. The group can be neutral or positively charged, depending upon the pH of the coating composition. In some embodiments, the radical Y includes an ammonium group. Suitable counter ions include, for example, carboxylates, halides, sulfate, and phosphate. For example, compounds of formula I can have a Y radical that contains an ammonium group; X₁ and X₂ can contain photoreactive groups that include aryl ketones. Such photoactivatable cross-linking agents include ethylenebis(4-benzoylbenzyldimethylammonium) salt; hexamethylenebis (4-benzoylbenzyldimethylammonium) salt; 1,4-bis(4-benzoylbenzyl)-1,4-dimethylpiperazinediium) salt, bis(4-benzoylbenzyl)hexamethylenetetraminediium salt, bis[2-(4-benzoylbenzyldimethylammonio)ethyl]-4-benzoylbenzylmethylammonium salt; 4,4-bis(4-benzoylbenzyl)morpholinium salt; ethylenebis[(2-(4-benzoylbenzyldimethylammonio)ethyl)-4-benzoylbenzylmethylammonium] salt; and 1,1,4,4-tetrakis(4-benzoylbenzyl)piperzinediium salt. See U.S. Pat. No. 5,714,360. The counter ion is typically a carboxylate ion or a halide. On one embodiment, the halide is bromide.

In other embodiments, the ionic photoactivatable cross-linking agent can be a compound having the formula:

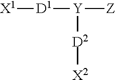

wherein X¹ includes a first photoreactive group; X² includes a second photoreactive group; Y includes a core molecule; Z includes at least one charged group; D¹ includes a first degradable linker; and D² includes a second degradable linker. Additional exemplary degradable ionic photoactivatable cross-linking agents are described in US Patent Application Publication US 2011/0144373 (Swan et al., “Water Soluble Degradable Crosslinker”), the disclosure of which is incorporated herein by reference.

In some aspects a non-ionic photoactivatable cross-linking agent can be used. In one embodiment, the non-ionic photoactivatable cross-linking agent has the formula XR₁R₂R₃R₄, where X is a chemical backbone, and R₁, R₂, R₃, and R₄ are radicals that include a latent photoreactive group. Exemplary non-ionic cross-linking agents are described, for example, in U.S. Pat. Nos. 5,414,075 and 5,637,460 (Swan et al., “Restrained Multifunctional Reagent for Surface Modification”). Chemically, the first and second photoreactive groups, and respective spacers, can be the same or different.

In other embodiments, the non-ionic photoactivatable cross-linking agent can be represented by the formula:

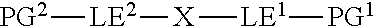

wherein PG¹ and PG² include, independently, one or more photoreactive groups, for example, an aryl ketone photoreactive group, including, but not limited to, aryl ketones such as acetophenone, benzophenone, anthraquinone, anthrone, anthrone-like heterocycles, their substituted derivatives or a combination thereof; LE¹ and LE² are, independently, linking elements, including, for example, segments that include urea, carbamate, or a combination thereof; and X represents a core molecule, which can be either polymeric or non-polymeric, including, but not limited to a hydrocarbon, including a hydrocarbon that is linear, branched, cyclic, or a combination thereof; aromatic, non-aromatic, or a combination thereof; monocyclic, polycyclic, carbocyclic, heterocyclic, or a combination thereof; benzene or a derivative thereof; or a combination thereof. Other non-ionic crosslinking agents are described, for example, in U.S. application Ser. No. 13/316,030 filed Dec. 9, 2011 (Publ. No. US 2012/0149934) (Kurdyumov, “Photocrosslinker”), the disclosure of which is incorporated herein by reference.

Further embodiments of non-ionic photoactivatable cross-linking agents can include, for example, those described in U.S. Provisional Application 61/494,724 filed Jun. 8, 2011 (now U.S. application Ser. No. 13/490,994) (Swan et al., “Photo-Vinyl Primers/Crosslinkers”), the disclosure of which is incorporated herein by reference. Exemplary cross-linking agents can include non-ionic photoactivatable cross-linking agents having the general formula R¹—X—R², wherein R¹ is a radical comprising a vinyl group, X is a radical comprising from about one to about twenty carbon atoms, and R² is a radical comprising a photoreactive group.

Some suitable cross-linking agents are those formed by a mixture of the chemical backbone molecule (such as pentaerythritol) and an excess of a derivative of the photoreactive group (such as 4-bromomethylbenzophenone). An exemplary product is tetrakis(4-benzoylbenzyl ether) of pentaerythritol (tetrakis(4-benzoylphenylmethoxymethyl)methane). See U.S. Pat. Nos. 5,414,075 and 5,637,460.

A single photoactivatable cross-linking agent or any combination of photoactivatable cross-linking agents can be used in forming the coating. In some embodiments, at least one nonionic cross-linking agent such as tetrakis(4-benzoylbenzyl ether) of pentaerythritol can be used with at least one ionic cross-linking agent. For example, at least one non-ionic photoactivatable cross-linking agent can be used with at least one cationic photoactivatable cross-linking agent such as an ethylenebis(4-benzoylbenzyldimethylammonium) salt or at least one anionic photoactivatable cross-linking agent such as 4,5-bis(4-benzoyl-phenylmethyleneoxy)benzene-1,3-disulfonic acid or salt. In another example, at least one nonionic cross-linking agent can be used with at least one cationic cross-linking agent and at least one anionic cross-linking agent. In yet another example, a least one cationic cross-linking agent can be used with at least one anionic cross-linking agent but without a non-ionic cross-linking agent.

An exemplary cross-linking agent is disodium 4,5-bis[(4-benzoylbenzyl)oxy]-1,3-benzenedisulfonate (DBDS). This reagent can be prepared by combining 4,5-Dihydroxylbenzyl-1,3-disulfonate (CHBDS) with 4-bromomethylbenzophenone (BMBP) in THF and sodium hydroxide, then refluxing and cooling the mixture followed by purification and recrystallization (also as described in U.S. Pat. No. 5,714,360, incorporated herein by reference).

A further exemplary cross-linking agent is ethylenebis (4-benzoylbenzyldimethylammonium) dibromide. This agent can be prepared as described in U.S. Pat. No. 5,714,360, the content of which is herein incorporated by reference.

Further cross-linking agents can include the cross-linking agents described in U.S. Publ. Pat. App. No. 2010/0274012 and U.S. Pat. No. 7,772,393 the content of all of which is herein incorporated by reference.

In some embodiments, cross-linking agents can include boron-containing linking agents including, but not limited to, the boron-containing linking agents disclosed in U.S. 61/666,516, entitled “Boron-Containing Linking Agents” by Kurdyumov et al., the content of which is herein incorporated by reference. By way of example, linking agents can include borate, borazine, or boronate groups and coatings and devices that incorporate such linking agents, along with related methods. In an embodiment, the linking agent includes a compound having the structure (I):

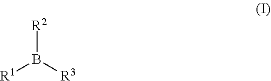

wherein R¹ is a radical comprising a photoreactive group; R² is selected from OH and a radical comprising a photoreactive group, an alkyl group and an aryl group; and R³ is selected from OH and a radical comprising a photoreactive group. In some embodiments the bonds B—R¹, B—R² and B—R³ can be chosen independently to be interrupted by a heteroatom, such as O, N, S, or mixtures thereof.

Additional agents for use with embodiments herein can include stilbene-based reactive compounds including, but not limited to, those disclosed in U.S. 61/736,436, entitled “Stilbene-Based Reactive Compounds, Polymeric Matrices Formed Therefrom, and Articles Visualizable by Fluorescence” by Kurdyumov et al., the content of which is herein incorporated by reference.

Additional photoreactive agents, cross-linking agents, hydrophilic coatings, and associated reagents are disclosed in US2011/0059874; US 2011/0046255; and US 2010/0198168, the content of all of which is herein incorporated by reference. Further exemplary cross-linking agents are described in U.S. Publ. Pat. App. No. 2011/0245367, the content of which is herein incorporated by reference in its entirety.

### Substrates

The substrate can be formed from any desirable material, or combination of materials, suitable for use within the body. In some embodiments the substrate is formed from compliant and flexible materials, such as elastomers (polymers with elastic properties). Exemplary elastomers can be formed from various polymers including polyurethanes and polyurethane copolymers, polyethylene, styrene-butadiene copolymers, polyisoprene, isobutylene-isoprene copolymers (butyl rubber), including halogenated butyl rubber, butadiene-styrene-acrylonitrile copolymers, silicone polymers, fluorosilicone polymers, polycarbonates, polyamides, polyesters, polyvinyl chloride, polyether-polyester copolymers, polyether-polyamide copolymers, and the like. The substrate can be made of a single elastomeric material, or a combination of materials.

Other materials for the substrate can include those formed of polymers, including oligomers, homopolymers, and copolymers resulting from either addition or condensation polymerizations. Examples of suitable addition polymers include, but are not limited to, acrylics such as those polymerized from methyl acrylate, methyl methacrylate, hydroxyethyl methacrylate, hydroxyethyl acrylate, acrylic acid, methacrylic acid, glyceryl acrylate, glyceryl methacrylate, methacrylamide, and acrylamide; vinyls such as ethylene, propylene, vinyl chloride, vinyl acetate, vinyl pyrrolidone, vinylidene difluoride, and styrene. Examples of condensation polymers include, but are not limited to, nylons such as polycaprolactam, polylauryl lactam, polyhexamethylene adipamide, and polyhexamethylene dodecanediamide, and also polyurethanes, polycarbonates, polyamides, polysulfones, poly(ethylene terephthalate), polydimethylsiloxanes, and polyetherketone.

Beyond polymers, and depending on the type of device, the substrate can also be formed of other inorganic materials such as metals (including metal foils and metal alloys), glass and ceramics.

Processes to modify substrates described above can include chemical modifications to improve performance characteristics of the substrate. Specific chemical processes that can be used include ozone treatment, chemical oxidation, acid chemical etching, base chemical etching, plasma treatment and corona treatment, surface grafting, thermally activated coating processes (both covalent and non-covalent) and surface modifications including coatings containing dopamine, tannic acid, plant polyphenols and other catechols or catechol containing derivatives of hydrophilic moieties. Additionally, processes to form substrates described above can include physical modifications for example, but not limited to, sand blasting and surface texturing (for example either during or after the molding process of polymers).

In some embodiments, a base coat layer can be used on top of the substrate. The base coat layer can provide for various functional properties. In some embodiments, a base coat layer herein can provide for improved adhesion of overlying layers to the substrate. In some embodiments, the base coat layer can be a tie layer.

### Medical Devices

It will be appreciated that embodiments herein include, and can be used in conjunction with, various types of medical devices including, but not limited to, various types of catheters, drug delivery devices such as drug eluting balloon catheters, drug-containing balloon catheters, stents, grafts, and the like.

Some embodiments described herein can be used in conjunction with balloon expandable flow diverters, and self-expanding flow diverters. Other embodiments can include uses in contact with angioplasty balloons (for example, but not limited to, percutaneous transluminal coronary angioplasty and percutaneous transluminal angioplasty). Yet other embodiments can include uses in conjunction with sinoplasty balloons for ENT treatments, urethral balloons and urethral stents for urological treatments and gastro-intestinal treatments (for example, devices used for colonoscopy). Hydrophobic active agent can be transferred to tissue from a balloon-like inflatable device or from a patch-like device. Other embodiments of the present disclosure can further be used in conjunction with micro-infusion catheter devices. In some embodiments, micro-infusion catheter devices can be used to target active agents to the renal sympathetic nerves to treat, for example, hypertension.

Other exemplary medical applications wherein embodiments of the present disclosure can be used further encompass treatments for bladder neck stenosis (e.g. subsequent to transurethral resection of the prostrate), laryngotrachial stenosis (e.g. in conjunction with serial endoscopic dilatation to treat subglottic stenosis, treatment of oral cancers and cold sores and bile duct stenosis (e.g. subsequent to pancreatic, hepatocellular of bile duct cancer). By way of further example, embodiments herein can be used in conjunction with drug applicators. Drug applicators can include those for use with various procedures, including surgical procedures, wherein active agents need to be applied to specific tissue locations. Examples can include, but are not limited to, drug applicators that can be used in orthopedic surgery in order to apply active agents to specific surfaces of bone, cartilage, ligaments, or other tissue through physical contact of the drug applicator with those tissues. Drug applicators can include, without limitation, hand-held drug applicators, drug patches, drug stamps, drug application disks, and the like.

It should be noted that, as used in this specification and the appended claims, the singular forms “a,” “an,” and “the” include plural referents unless the content clearly dictates otherwise. Thus, for example, reference to a composition containing “a compound” includes a mixture of two or more compounds. It should also be noted that the term “or” is generally employed in its sense including “and/or” unless the content clearly dictates otherwise.

It should also be noted that, as used in this specification and the appended claims, the phrase “configured” describes a system, apparatus, or other structure that is constructed or configured to perform a particular task or adopt a particular configuration. The phrase “configured” can be used interchangeably with other similar phrases such as arranged and configured, constructed and arranged, constructed, manufactured and arranged, and the like.

All publications and patent applications in this specification are indicative of the level of ordinary skill in the art to which this invention pertains. All publications and patent applications are herein incorporated by reference to the same extent as if each individual publication or patent application was specifically and individually indicated by reference.

As used herein, the recitation of numerical ranges by endpoints shall include all numbers subsumed within that range (e.g., 2 to 8 includes 2.1, 2.8, 5.3, 7, etc.).

The headings used herein are provided for consistency with suggestions under 37 CFR 1.77 or otherwise to provide organizational cues. These headings shall not be viewed to limit or characterize the invention(s) set out in any claims that may issue from this disclosure. As an example, although the headings refer to a “Field,” such claims should not be limited by the language chosen under this heading to describe the so-called technical field. Further, a description of a technology in the “Background” is not an admission that technology is prior art to any invention(s) in this disclosure. Neither is the “Summary” to be considered as a characterization of the invention(s) set forth in issued claims.

The embodiments described herein are not intended to be exhaustive or to limit the invention to the precise forms disclosed in the following detailed description. Rather, the embodiments are chosen and described so that others skilled in the art can appreciate and understand the principles and practices. As such, aspects have been described with reference to various specific and preferred embodiments and techniques. However, it should be understood that many variations and modifications may be made while remaining within the spirit and scope herein.

1. A coated medical device comprising:
a substrate; and
a lubricious coating, the lubricious coating comprising
an outer protective layer, the outer protective layer comprising long polymer chains, wherein the long polymer chains include a bonded end and a mobile free end; and
wherein the lubricious coating is disposed on the substrate.

2. The coated medical device of claim 1, wherein the long polymer chains have an average chain length greater than 100 kDa.

3. The coated medical device of claim 1, wherein the long polymer chains have an average chain length from 100 to 300 kDa.

4-5. (canceled)

6. The coated medical device of claim 1, the long polymer chains comprising at least one selected from the group consisting of a comb structure, a bottle brush structure, a star structure, a hyperbranched structure, a mucin structure, and a dendritic structure.

7. The coated medical device of claim 1, wherein the long polymer chains are uncross-linked.

8. The coated medical device of claim 1, the lubricious coating further comprising an underlying hydrogel layer.

9. The coated medical device of claim 8, the hydrogel layer comprising one or more of polyvinylpyrrolidone (PVP), a photo-reactive PVP, polyacrylamide (PA), and a photo-reactive PA.

10. The coated medical device of claim 8, wherein the outer protective layer exhibits a coefficient of friction less than the hydrogel layer.

11. The coated medical device of claim 8, the hydrogel layer comprising:
a first sublayer; and
a second sublayer.

12. The coated medical device of claim 11, wherein the second sublayer is crosslinked to a greater extent than the first sublayer.

13. The coated medical device of claim 8, the lubricious coating further comprising a base coat.

14-16. (canceled)

17. The coated medical device of claim 1, the long polymer chains comprising at least one selected from the group consisting of a polyvinylpyrrolidone, a polyvinylalcohol, a polyacrylamide, a polyethyleneglycol, a polysulfobetaine, a phosphoryl choline capped polymer, and a poloxamer.

18. The coated medical device of claim 1, the long polymer chains comprising a polyzwitterion.

19. The coated medical device of claim 1, the long polymer chains comprising a zwitterionic subunit, wherein the zwitterionic subunit disposed on an exterior portion of the long polymer chains.

20. The coated medical device of claim 1, the long polymer chains comprising a zwitterionic subunit, wherein the zwitterionic subunit is disposed on an end of the long polymer chains.

21. The coated medical device of claim 1, the long polymer chains comprising a non-ionic backbone chain.

22. The coated medical device of claim 1, wherein the long polymer chains are grafted onto an underlying surface or at least partially embedded within and crosslinked into an underlying layer.

23-24. (canceled)

25. The coated medical device of claim 1, the outer protective layer further comprising non-covalently bonded long polymer chains, wherein the non-covalently bonded long polymer chains are configured to elute from the lubricious coating.

26. The coated medical device of claim 1, wherein the long polymer chains include a covalently bonded end and a mobile free end.

27-32. (canceled)

33. A coated medical device comprising:
a substrate; and
a lubricious coating, the lubricious coating comprising
a hydrogel layer; and
long polymer chains, wherein the long polymer chains are disposed at least partially within the lubricious coating.

34-136. (canceled)
